(19) United States
(12) Patent Application Publication
(10) Pub. No.: US 2004/0265836 A1
(43) Pub. Date: Dec. 30, 2004

(54) Methods for the identification of inhibitors of C4 Long Chain Base Hydroxylase activity in plants

(76) Inventors: M. DeZwaan Todd, Apex, NC (US); Lining Guo, Chapel Hill, NC (US); Keith Davis, Durham, NC (US); Adel Zayed, Durham, NC (US); Susanne Kjemtrup, Chapel Hill, NC (US); Douglas Boyes, Chapel Hill, NC (US); Brenda Warrick, Cary, NC (US); Joseph Cameron Mitchell, Chapel Hill, NC (US); Robert Ascenzi, Cary, NC (US); Carol Hamilton, Apex, NC (US); Jeffrey Woessner, Hillsborough, NC (US); Jorn Gorlach, Manchester, NJ (US); Neil Hoffman, Chapel Hill, NC (US); Rao Mulpuri, Apex, NC (US)

Correspondence Address: Paradigm Genetics, Inc., Patent and Trademark Department, 108 Alexander Drive, P.O. Box 14528, Research Triangle Park, NC, 27709, US

(21) Appl. No.: 10/610,156

(22) Filed: Jun. 30, 2003

(51) Int. Cl. C12Q 1/68

(52) U.S. Cl. 435/6

(57) ABSTRACT

The present inventors have discovered that LCBH is essential for plant growth. Specifically, the inhibition of LCBH gene expression in Arabidopsis results in reduced growth and abnormal development. Thus, LCBH is useful as a target for the identification of herbicides. Accordingly, the present invention provides methods for the identification of herbicides by measuring the activity of a LCBH in the presence and absence of a compound, where an alteration of LCBH activity in the presence of the compound indicates the compound as a candidate for a herbicide.

### FIELD OF THE INVENTION

The invention relates generally to plant molecular biology. In particular, the invention relates to methods for the identification of herbicides.

### BACKGROUND OF THE INVENTION

The traditional approach to herbicide development is to spray chemicals, produced in milligram or greater quantity, on plants and then to monitor plant growth. While the spray and observe approach has resulted in the identification of commercially important herbicides, rising costs, and efficacy and safety concerns are challenging its future productivity. Accordingly, there is a need to identify herbicide targets so that compound libraries can be screened for herbicidal activity in higher through-put in vitro or cell-based assays. Inhibitors of the identified targets can then be selected and confirmed as having herbicidal activity using conventional assays.

The present invention provides compositions and methodologies for C4-Long Chain Base Hydroxylase (LCBH) as a target for the identification of plant growth regulators, especially herbicide compounds, in plants. LCBH, also known as sphingolipid C4-hydroxylase, catalyzes C4-hydroxylation of long chain bases in plant sphingolipids, generating phytosphinganine. Sphingolipids are ubiquitous membrane components in eukaryotic cells and a few bacteria, and also are involved in signal transduction and other cellular processes. Little is known of the role of sphingolipids in plants (Sperling P. et al., 494 FEBS Lett. 90-4 (2001)). Two genes encoding C4-LCBH isozymes have been identified in Arabidopsis thaliana. Id. The Arabidopsis C4-LCBH isozymes share a high degree of sequence conservation with yeast di-iron-binding enzymes involved in oxygen-dependent lipid modification, Sur2p (also known as Syr2) and Erg3p. It has been shown that the Sur2 gene is not essential for growth, as no defect on vegetative growth or stress resistance was observed in a yeast null mutant strain (Grilley M. M. et al., 273 J. Biol. Chem. 11062-8 (1998)). Sur2/Syr2 is required for 4-hydroxylation of sphingoid bases in Saccharomyces cerevisiae, an activity that is necessary for growth inhibition by syringomycin E. Id.

### SUMMARY OF THE INVENTION

The present inventors have discovered that antisense suppression of C4-Long Chain Base Hydroxylase (LCBH) in Arabidopsis thaliana results in stunted seedlings with asymmetric cotyledons and necrotic patches. Thus, the LCBH is essential for normal plant development and growth, and is useful as a target for the identification of herbicides.

Accordingly, in one embodiment the present invention provides methods for identifying a compound as a candidate for a herbicide, comprising: measuring the activity of a LCBH protein in the presence and absence of a compound, wherein an alteration of the LCBH activity in the presence of the compound indicates the compound as a candidate for a herbicide.

In another embodiment, the present invention provides a method for the identification of a compound that inhibits LCBH activity as a candidate for a herbicide, comprising: measuring growth of a sur2 strain of mutant yeast cells expressing a heterologous LCBH polypeptide in a media containing syringomycin E, in the presence and absence of a compound, wherein the heterologous LCBH polypeptide confers on the mutant yeast cells a sensitivity to syringomycin E and the syringomycin E is present at an amount sufficient to suppress growth of the yeast cells; and comparing growth of the mutant yeast cells in the presence and absence of the compound, wherein an increase in growth in the presence, relative to the absence, of the compound indicates the compound as a candidate for a herbicide.

In another embodiment, the present invention provides a method for the identification of a compound as a candidate for a herbicide, comprising: contacting a LCBH polypeptide with a compound; and detecting the presence or absence of binding between the compound and the LCBH polypeptide, wherein binding indicates that the compound is a candidate for a herbicide.

In another embodiment, the invention provides a method for identifying a compound as a candidate for a herbicide, comprising: measuring the expression of a LCBH in a plant, or tissue thereof, in the presence and absence of a compound; and comparing the expression of the LCBH in the presence and absence of the compound, wherein an altered expression in the presence of the compound indicates that the compound is a candidate for a herbicide.

### DETAILED DESCRIPTION OF THE INVENTION

The term “bDNA” refers to branched DNA.

As used herein, the term “cDNA” means complementary deoxyribonucleic acid.

As used herein, the term “ELISA” means enzyme-linked immunosorbent assay.

As used herein, the term “GUS” means β-glucouronidase.

The term “herbicide,” as used herein, refers to a compound useful for killing or suppressing the growth of at least one plant, plant cell, plant tissue or seed.

The phrase “heterologous LCBH protein,” as used herein, refers to any LCBH polypeptide that is encoded by a nucleic acid molecule that has been transformed or introduced into mutant yeast cells being used in a cell based assay for identifying inhibitors of LCBH activity.

The term “inhibitor,” as used herein, refers to a chemical substance that inactivates the enzymatic activity of LCBH or substantially reduces the level of enzymatic activity, wherein “substantially” means a reduction at least as great as the standard deviation for a measurement, preferably a reduction by 50%, more preferably a reduction of at least one magnitude, i.e. to 10%. The inhibitor may function by interacting directly with the enzyme, a cofactor of the enzyme, the substrate of the enzyme, or any combination thereof.

A polynucleotide is “introduced” into a plant cell by any means, including transfection, transformation or transduction, electroporation, particle bombardment, agroinfection and the like. The introduced polynucleotide is maintained in the cell stably if it is incorporated into a non-chromosomal autonomous replicon or integrated into the plant chromosome. Alternatively, the introduced polynucleotide is present on an extra-chromosomal non-replicating vector and be transiently expressed or transiently active.

As used herein, the terms “long chain base hydroxylase (LCBH) protein,” “C4-long chain base hydroxylase (C4-LCBH) protein,” and “sphingolipid C4-hydroxylase” are interchangeable, and refer to an enzyme that catalyzes C4-hydroxylation of long chain bases in plant sphingolipids, generating phytosphinganine. As used herein, the term “LCBH protein” means either a nucleic acid encoding a polypeptide or a polypeptide, wherein the polypeptide has at least 40%, 45%, 50%, 55%, 60%, 65%, 70%, 75%, 80%, 85%, 90%, 91%, 92%, 93%, 94%, 95%, 96%, 97%, 98%, or 99% sequence conservation or each integer unit of sequence conservation from 40-100% in ascending order to Arabidopsis LCBH protein (SEQ ID NO:2) and at least 10%, 25%, 50%, 75%, 80%, 90%, 95%, or 99% activity or each integer unit of activity from 10-100% in ascending order of the activity of Arabidopsis LCBH protein (SEQ ID NO:2). Examples of LCBH proteins include, but are not limited to, LCBH from Oryza sativa and LCBH from Lupinus albus.

As used herein, the term “PCR” means polymerase chain reaction.

The “percent (%) sequence conservation” between two polynucleotide or two polypeptide sequences is determined according to the either the BLAST program (Basic Local Alignment Search Tool, Altschul and Gish (1996) Meth Enzymol 266: 460-480; Altschul (1990) J Mol Biol 215: 403-410) or using Smith Waterman Alignment (Smith and Waterman (1981) Adv Appl Math 2:482) using the default settings and the version current at the time of filing).

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “plant LCBH protein” is meant a protein found in at least one plant, and which catalyzes LCBH activity. The LCBH is from any plant, including monocots, dicots, C3 plants, C4 plants and/or plants that are classified as neither C3 nor C4 plants.

By “polypeptide” is meant a chain of at least four amino acids joined by peptide bonds. The chain is linear, branched, circular or combinations thereof. The polypeptides may contain amino acid analogs and other modifications, including, but not limited to glycosylated or phosphorylated residues.

The present inventors have discovered that inhibition of LCBH gene expression in Arabidopsis thaliana results in reduced growth and abnormal development. The present result indicates LCBH proteins as useful targets for the identification of herbicides. Accordingly, the present invention provides methods for identifying compounds that inhibit LCBH protein activity. Such methods include binding assays, activity assays and assays for LCBH gene expression. The compounds identified by the methods of the invention are useful as herbicides.

A cDNA encoding an Arabidopsis LCBH is set forth in SEQ ID NO:1. The protein encoded by the Arabidopsis LCBH cDNA is set forth in SEQ ID NO:2. Arabidopsis LCBH displays a high degree of sequence conservation with LCBH from Oryza sativa (Accession NO: BAB63743) and LCBH from Lupinus albus (Accession NO: BAA82130). In addition to the LCBH proteins described above, numerous other LCBH polypeptides are useful in the methods of the invention. For example, LCBH proteins having the amino acid sequence of a naturally occurring LCBH found in a plant, animal or microorganism are useful in the invention. In another embodiment of the invention, the LCBH polypeptide has an amino acid sequence derived from a naturally occurring sequence. In another embodiment the LCBH is a plant LCBH protein. In another embodiment the LCBH is an Arabidopsis LCBH protein. In one embodiment, the LCBH is an Arabidopsis LCBH protein. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii.

In various embodiments, the LCBH can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

The invention further provides LCBH polypeptides that are N-terminal truncated derivatives of naturally occurring LCBH proteins. For example, a nucleic acid molecule encoding an N-terminal 108 amino acid truncated Arabidopsis LCBH is set forth in SEQ ID NO:3. The truncated polypeptide encoded by SEQ ID NO:3 is set forth in SEQ ID NO:4. Polypeptides consisting essentially of SEQ ID NO:2 are also useful in the methods of the invention. For the purposes of the present invention, a polypeptide consisting essentially of SEQ ID NO:2 has at least 90% sequence conservation with SEQ ID NO:2 and at least 10% of the activity of SEQ ID NO:2. A polypeptide consisting essentially of SEQ ID NO:2 has at least 90%, 91%, 92%, 93%, 94%, 95%, 96%, 97%, 98%, or 99% sequence conservation with SEQ ID NO:2 and at least 25%, 50%, 75%, or 90% of the activity of SEQ ID NO:2.

Examples of polypeptides consisting essentially of SEQ ID NO:2 include, but are not limited to, polypeptides having the amino acid sequence of SEQ ID NO:2 with the exception that one or more of the amino acids are substituted with structurally similar amino acids providing a “conservative amino acid substitution.” Conservative amino acid substitutions are well known to those of skill in the art. Particular examples of polypeptides consisting essentially of SEQ ID NO:2 include polypeptides having 1, 2, or 3 conservative amino acid substitutions relative to SEQ ID NO:4.

Other examples of polypeptides consisting essentially of SEQ ID NO:2 include polypeptides having the sequence of SEQ ID NO:2, but with truncations at either or both the 3′ and the 5′ end. For example, polypeptides consisting essentially of SEQ ID NO:2 include polypeptides having 1, 2, or 3 amino acids residues removed from either or both 3′ and 5′ ends relative to SEQ ID NO:2. Additional examples of polypeptides consisting essentially of SEQ ID NO:2 include polypeptides having 1, 2, 3, 4, 5, 6, 7, 8, 9, or 10 fewer amino acids residues truncated from the N-terminus relative to SEQ ID NO:2. In addition, LCBH polypeptides consisting essentially of SEQ ID NO:2 can be fusion proteins, such as SEQ ID NO:2 fused to other polypeptide or amino acid sequences to aid in secretion and/or isolation as is known to those of ordinary skill in the art.

LCBH polypeptides having at least 40% sequence conservation with SEQ ID NO:2 are also useful in the methods of the invention. In one embodiment, the sequence conservation is at least 40%, 45%, 50%, 55%, 60%, 65%, 70%, 75%, 80%, 85%, 90%, 91%, 92%, 93%, 94%, 95%, 96%, 97%, 98% or 99%, or any integer from 40-100% sequence conservation in ascending order with SEQ ID NO:2. In addition, it is preferred that LCBH polypeptides of the invention have at least 10% of the activity of SEQ ID NO:2. LCBH polypeptides of the invention have at least 10%, 15%, 20%, 25%, 30%, 35%, 40%, 45%, 50%, 55%, 60%, 65%, 70%, 75%, 80%, 85% or at least 90% of the activity of SEQ ID NO:2.

Fragments of a LCBH polypeptide are useful in the methods of the invention. In one embodiment, the LCBH fragments include an intact or nearly intact epitope that occurs on the biologically active wild-type LCBH protein. For example, the fragments comprise at least 10 consecutive amino acids of SEQ ID NO:2. The fragments comprise at least 15, 20, 25, 30, 35, 40, 50, 60, 70, 80, 90, 100, 125, 150, 175, 200, 225 or at least 255 consecutive amino acid residues of SEQ ID NO:2. Polypeptides comprising at least 50 amino acids having at least 50%, 60%, 70%, 80%, 90%, 95%, 96%, 97%, 98% or 99% sequence conservation with at least 50 consecutive amino acid residues of SEQ ID NO:2 are also useful in the methods of the invention. In one embodiment, the fragment is from an Arabidopsis LCBH protein. In one embodiment, the fragment contains an amino acid sequence conserved among plant LCBH sequences.

Enzymatically active fragments of Arabidopsis LCBH set forth in SEQ ID NO:2 are also useful in the methods of the invention. For example, enzymatically active polypeptides comprising at least 50 consecutive amino acid residues and at least 10% of the activity of Arabidopsis LCBH set forth in SEQ ID NO:2 are useful in the methods of the invention. The fragments comprise at least 15, 20, 25, 30, 35, 40, 50, 60, 70, 80, 90, 100, 125, 150, 175, 200, 225 or at least 255 consecutive amino acid residues SEQ ID NO:2. In addition, enzymatically active fragments of LCBH proteins useful in the methods of the invention, include polypeptides comprising at least 50 amino acids having at least 10% of the activity of SEQ ID NO:2 and at least 50%, 60%, 70%, 80%, 90%, 95%, 96%, 97%, 98% or 99% sequence conservation with at least 50 consecutive amino acid residues of SEQ ID NO:2. Most preferably, the enzymatically active polypeptides comprise at least 50 amino acids, have at least 50% sequence conservation with at least 50 consecutive amino acid residues of SEQ ID NO:2 and at least 25%, 75% or at least 90% of the activity thereof.

Thus, in one embodiment, the invention provides binding assays to identify compounds that are useful as herbicides. One method for identifying a compound as a candidate for a herbicide, comprises: contacting a compound with a LCBH polypeptide selected from the group consisting of: a polypeptide set forth in SEQ ID NO:2 or SEQ ID NO:4; a LCBH polypeptide consisting essentially of SEQ ID NO:2; a polypeptide comprising at least 10 consecutive amino acids of SEQ ID NO:2; a LCBH polypeptide having at least 50% sequence conservation with SEQ ID NO:2; and a LCBH polypeptide comprising at least 50 amino acids having at least 50% sequence conservation with at least 50 consecutive amino acid residues of SEQ ID NO:2; and detecting the presence and/or absence of binding between the compound and the polypeptide, wherein binding indicates that the compound is a candidate for a herbicide.

Any technique for detecting the binding of a ligand to its target is useful in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a LCBH protein or a fragment or variant thereof, the unbound protein is removed and the bound LCBH is detected. In a preferred embodiment, bound LCBH is detected using a labeled binding partner, such as a labeled antibody. In a variation of this assay, LCBH is labeled prior to contacting the immobilized candidate ligands. Preferred labels include fluorescent or radioactive moieties. In other embodiments of the invention, detection methods include fluorescence correlation spectroscopy (FCS) and FCS-related confocal nanofluorimetric methods.

In another embodiment of the invention, compounds are tested as candidate herbicides based on ability to inhibit LCBH enzyme activity. The compounds are tested using either in vitro or cell based enzyme assays. Thus, in one embodiment, the invention provides a method for the identification of a compound as a herbicide, comprising: measuring the activity of a LCBH in the presence and absence of a compound, wherein an alteration of the LCBH activity in the presence of the compound indicates the compound as a candidate for a herbicide.

One method for identifying a compound as a candidate for a herbicide, comprises: measuring the activity of a LCBH in the presence and absence of a compound, wherein an alteration of the LCBH activity in the presence of the compound indicates the compound as a candidate for a herbicide. The LCBH polypeptide is selected from the group consisting of: a polypeptide set forth in SEQ ID NO:2 or SEQ ID NO:4; a LCBH polypeptide consisting essentially of SEQ ID NO:2; a LCBH polypeptide comprising at least 50 consecutive amino acids of SEQ ID NO:2 and having at least 10% of the activity of SEQ ID NO:2; a LCBH polypeptide having at least 50% sequence conservation with SEQ ID NO:2 and having at least 10% of the activity of SEQ ID NO:2; and a LCBH polypeptide comprising at least 50 amino acids having at least 50% sequence conservation with SEQ ID NO:2 and having at least 10% of the activity of SEQ ID NO:2.

Methods for measuring LCBH activity include the use of in vitro enzymatic assays in which the disappearance of a substrate or the appearance of a product is directly or indirectly detected. Methods for measuring the progression of the LCBH enzymatic reaction and/or a change in the concentration of one or more substrates and/or products, include spectrophotometry, fluorimetry, mass spectroscopy, thin layer chromatography (TLC) and reverse phase HPLC. LCBH activity utilizes NAD[P]H which can be measured by exciting at 340 nm and recording the emission at 465 nm. A change in emission at 465 mm is useful for detecting compounds that bind to LCBH as well as those that inhibit LCBH activity.

For in vitro assays, LCBH polypeptides, and derivatives thereof, are isolated from a plant or may be recombinantly produced in and isolated from an archael, bacterial, fungal, or other eukaryotic cell culture. Preferably the proteins are produced using an E. coli, yeast, or baculovirus expression system. In one case, the LCBH protein is expressed as a soluble product with secretory signal removed and in another case the LCBH protein is expressed and isolated as a microsomal preparation in a yeast or baculovirus system. Methods for the purification of LCBH proteins and polypeptides are known to those or ordinary skill in the art including, for example, the use of either N- or C-terminal 6×His tags.

In addition to the LCBH orthologs described supra, Arabidopsis LCBH also shares sequence conservation with the LCBH from Saccharomyces cerevisiae encoded by the SUR2 gene (Accession NO: AAB41115). SUR2 was identified in a yeast genetic screen for mutants that are resistant to the phytotoxin syringomycin E (Cliften & Wang et al., 142 Microbiology 477-84 (1996)). Syringomycin E is a lipodepsinonapeptide that is produced by the plant pathogenic bacterium Pseudomonas syringae pv. syringae (Bender & Alarcon-Chaidez et al., 62 Microbiol. Mol. Biol. Rev. (1999)). Wild-type yeast strains are sensitive to syringomycin E while sur2 mutants are able to grow in the presence of this compound.

Accordingly, the ability of a compound to inhibit LCBH activity can be detected using cell-based assays in which a heterologous LCBH polypeptide restores the Syringomycin E growth inhibition to a yeast Sur2 mutant strain. The Sur2 mutant strain grows normally in the presence of Syringomycin E and expression of a heterologous LCBH protein in the Sur2 background restores the growth inhibition. LCBH inhibitors are identified by their ability to confer growth on the heterologous LCBH protein-expressing Sur2 mutant. The phrase “heterologous LCBH protein” is herein intended to mean any LCBH polypeptide that is encoded by a nucleic acid molecule that has been transformed or introduced into the mutant yeast cells.

Thus, in one embodiment, the invention provides methods for the identification of compounds that inhibit LCBH activity, comprising: measuring cell growth of a sur2 strain of mutant yeast cells expressing a heterologous LCBH polypeptide in a media containing syringomycin E in the presence and absence of a compound, wherein the heterologous LCBH polypeptide confers on the mutant yeast cells a sensitivity to syringomycin E and the syringomycin E is present at an amount sufficient to suppress growth of the mutant yeast cells; and comparing the cell growth measurement in the presence and absence of the compound, wherein an increase in growth in the presence, relative to the absence, of the compound indicates the compound as a candidate for a herbicide.

In one embodiment of the invention, the heterologous LCBH is the polypeptide set forth in SEQ ID NO:2. In another embodiment, the LCBH is the polypeptide set forth in SEQ ID NO:4. In another embodiment, the LCBH is a polypeptide consisting essentially of SEQ ID NO:2. In another embodiment, the LCBH is an Arabidopsis LCBH polypeptide. In another embodiment, the LCBH is a plant LCBH protein.

In other embodiments of the invention, the LCBH is a polypeptide selected from the group consisting of: a polypeptide having at least 50% sequence conservation with SEQ ID NO:2 and having at least 10% of the activity thereof; a polypeptide comprising at least 50 consecutive amino acids of SEQ ID NO:2 and having at least 10% of the activity thereof; and a polypeptide comprising at least 50 amino acids, having at least 50% sequence conservation with SEQ ID NO:2 and having at least 10% of the activity thereof.

As an alternative to cell-based assays, the invention also provides plant-based assays. In one embodiment, the invention provides a method for identifying a compound as a candidate for a herbicide, comprising: a) measuring the expression or activity of a LCBH in a plant, or tissue thereof, in the absence of a compound; b) measuring the expression or activity of the LCBH in the plant, or tissue thereof, in the presence of the compound; and c) comparing the expression or activity of the LCBH in steps (a) and (b), wherein an altered expression or activity in the presence of the compound indicates that the compound is a candidate for a herbicide. In one embodiment, the plant or tissue thereof is Arabidopsis thaliana.

In the methods of the invention, expression of a LCBH in a plant, or tissue thereof, is measured by detecting the LCBH primary transcript or mRNA, LCBH polypeptide or LCBH enzymatic activity. Methods for detecting the expression of RNA and proteins are known to those skilled in the art. (See, for example, Current Protocols in Molecular Biology, Ausubel et al., eds., Greene Publishing and Wiley-Interscience, New York, 1995). However, the method of detection is not critical to the invention. Methods for detecting LCBH RNA include, but are not limited to, amplification assays such as quantitative PCR, and/or hybridization assays such as Northern analysis, dot blots, slot blots, in-situ hybridization, transcriptional fusions using a LCBH promoter fused to a reporter gene, bDNA assays, and microarray assays.

Methods for detecting protein expression include, but are not limited to, immunodetection methods such as Western blots, His Tag and ELISA assays, polyacrylamide gel electrophoresis, mass spectroscopy, and enzymatic assays. Also, any reporter gene system is useful to detect LCBH protein expression. For detection using gene reporter systems, a polynucleotide encoding a reporter protein is fused in frame with LCBH protein, so as to produce a chimeric polypeptide. Methods for using reporter systems are known to those skilled in the art. Examples of reporter genes include, but are not limited to, chloramphenicol acetyltransferase (Gorman et al. (1982) Mol Cell Biol 2: 1104; Prost et al. (1986) Gene 45: 107-111), β-galactosidase (Nolan et al. (1988) Proc Natl Acad Sci USA 85: 2603-2607), alkaline phosphatase (Berger et al. (1988) Gene 66: 10), luciferase (De Wet et al. (1987) Mol Cell Biol 7: 725-737), β-glucuronidase (GUS), fluorescent proteins, chromogenic proteins and the like.

Chemicals, compounds, or compositions identified by the above methods as modulators of LCBH expression or activity are useful for controlling plant growth. For example, compounds that inhibit plant growth are applied to a plant or expressed in a plant to prevent plant growth. Thus, the invention provides a method for inhibiting plant growth, comprising contacting a plant with a compound identified by the methods of the invention as having herbicidal activity. Compounds are tested by direct application to a plant or plant cell, or expressing it therein, and monitoring the plant or plant cell for changes or decreases in growth, development, viability or alterations in gene expression. A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be decreased by at least 40%. More preferably, the growth or viability will be decreased by at least 50%, 75%, or at least 90% or more. Methods for measuring plant growth and cell viability are known to those skilled in the art. It is possible that a candidate compound may have herbicidal activity only for certain plants or certain plant species.

Herbicides and herbicide candidates identified by the methods of the invention are useful for controlling the growth of undesired plants, including including monocots, dicots, C3 plants, C4 plants, and plants that are neither C3 nor C4 plants. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### EXPERIMENTAL

Plant Growth Conditions

Unless, otherwise indicated, all plants were grown in Scotts Metro-Mix™ soil (the Scotts Company) or a similar soil mixture in an environmental growth room at 22° C., 65% humidity, 65% humidity and a light intensity of ˜100 μ-E m⁻²s⁻¹ supplied over 16 hour day period.

Seed Sterilization

All seeds were surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% Clorox and 0.5% SDS solution and place on rotisserie for 10 minutes.

5. Carefully remove bleach/SDS solution.

6. Fill each tube with 1 ml sterile dl H₂O; seeds should be stirred up by pipetting of water into tube. Carefully remove water. Repeat 3 to 5 times to ensure removal of Clorox/SDS solution.

7. Fill each tube with enough sterile dl H₂O for seed plating (˜200-400 μl). Cap tube until ready to begin seed plating.

Plate Growth Assays

Surface sterilized seeds were sown onto plate containing 40 ml half strength sterile MS (Murashige and Skoog, no sucrose) medium and 1% Phytagel using the following protocol:

1. Using pipette man and 200 μl tip, carefully fill tip with seed solution. Place 10 seeds across the top of the plate, about ¼ inch down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22 and 20° C., respectively, 65% humidity and a light intensity of ˜100 μ-E m⁻²s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant Expressing the Driver

The “Driver” is an artificial transcription factor comprising a chimera of the DNA-binding domain of the yeast GAL4 protein (amino acid residues 1-147) fused to two tandem activation domains of herpes simplex virus protein VP16 (amino acid residues 413-490). Schwechheimer et al. (1998) Plant Mol Biol 36:195-204. This chimeric driver is a transcriptional activator specific for promoters having GAL4 binding sites. Expression of the driver is controlled by two tandem copies of the constitutive CaMV 35S promoter.

The driver expression cassette was introduced into Arabidopsis thaliana by agroinfection. Transgenic plants that stably expressed the driver transcription factor were obtained.

### EXAMPLE 2

### Construction of LCBH Antisense Expression Cassettes in a Binary Vector

A fragment of the Arabidopsis thaliana cDNA corresponding to SEQ ID NO:1 was ligated into the PacI/AscI sites of an E. coli/Agrobacterium binary vector in the antisense orientation to yield an antisense expression cassette and a constitutive chemical resistance expression cassette located between right and left T-DNA borders. In this construct, transcription of the antisense RNA is controlled by an artificial promoter active only in the presence of the driver transcription factor described above. The artificial promoter contains four contiguous binding sites for the GAL4 transcriptional activator upstream of a minimal promoter comprising a TATA box. The ligated DNA was transformed into E. coli. Kanamycin resistant clones were selected and purified. DNA was isolated from each clone and characterized by PCR and sequence analysis confirming the presence of the antisense expression cassette.

### EXAMPLE 3

### Transformation of Agrobacterium with the LCBH Antisense Expression Cassette

The binary vector described in Example 2 was transformed into Agrobacterium tumefaciens by electroporation. Transformed Agrobacterium colonies were isolated using chemical selection. DNA was prepared from purified resistant colonies and the inserts were amplified by PCR and sequenced to confirm sequence and orientation.

### EXAMPLE 4

### Construction of Arabidopsis LCBH Antisense Target Plants

The LCBH antisense expression cassette was introduced into Arabidopsis thaliana wild-type plants by the following method. Five days prior to agroinfection, the primary inflorescence of Arabidopsis thaliana plants grown in 2.5 inch pots were clipped to enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) was inoculated with a clonal glycerol stock of Agrobacterium carrying the desired DNA. The cultures were incubated overnight at 28° C. at 250 rpm until the cells reached stationary phase. The following morning, 200 ml LB in a 500 ml flask was inoculated with 500 μl of the overnight culture and the cells were grown to stationary phase by overnight incubation at 28° C. at 250 rpm. The cells were pelleted by centrifugation at 8000 rpm for 5 minutes. The supernatant was removed and excess media was removed by setting the centrifuge bottles upside down on a paper towel for several minutes. The cells were then resuspended in 500 ml infiltration medium (autoclaved 5% sucrose) and 250 μl/L Silwet L-77™ (84% polyalkyleneoxide modified heptamethyltrisiloxane and 16% allyloxypolyethyleneglycol methyl ether), and transferred to a one liter beaker.

The previously clipped Arabidopsis plants were dipped into the Agrobacterium suspension so that all above ground parts were immersed and agitated gently for 10 seconds. The dipped plants were then covered with a tall clear plastic dome to maintain the humidity, and returned to the growth room. The following day, the dome was removed and the plants were grown under normal light conditions until mature seeds were produced. Mature seeds were collected and stored desiccated at 4° C.

Transgenic Arabidopsis T1 seedlings were selected. Approximately 70 mg seeds from an agrotransformed plant were mixed approximately 4:1 with sand and placed in a 2 ml screw cap cryo vial. One vial of seeds was then sown in a cell of an 8 cell flat. The flat was covered with a dome, stored at 4° C. for 3 days, and then transferred to a growth room. The domes were removed when the seedlings first emerged. After the emergence of the first primary leaves, the flat was sprayed uniformly with a herbicide corresponding to the chemical resistance marker plus 0.005% Silwet (50 μl/L) until the leaves were completely wetted. The spraying was repeated for the following two days.

Ten days after the first spraying resistant plants were transplanted to 2.5 inch round pots containing moistened sterile potting soil. The transplants were then sprayed with herbicide and returned to the growth room. The herbicide resistant plants represented stably transformed Ti plants.

### EXAMPLE 5

### Effect of LCBH Antisense Expression in Arabidopsis Seedlings

The T1 LCBH antisense target plants from the transformed plant lines obtained in Example 4 were crossed with the Arabidopsis transgenic driver line described above. The resulting F1 seeds were then subjected to a plate assay to observe seedling growth over a 2-week period. Seedlings were inspected for growth and development. Antisense expression of the LCBH gene resulted in significantly impaired growth and/or asymmetric and chlorotic cotyledons, indicating that the LCBH gene is an essential gene for normal plant growth and development. Six of the nine transgenic plants showed the growth abnormalities.

### EXAMPLE 6

### Cloning and Expression of Full-Length and Amino-Terminal Truncated Derivatives of Arabidopsis LCBH in Yeast

Full-length A. thaliana C4-LCBH sequence (gb:) was analyzed with TargetP (v. 1.01) software program. TargetP predicts the subcellular location of eukaryotic protein sequences based on the predicted presence of amino-terminal presequences corresponding to chloroplast transit peptides, mitochondrial targeting peptides, or secretory pathway signal peptides, and also provides a potential cleavage site for sequences predicted to contain these targeting signals. The results of this analysis predicted a 108 amino acid amino-terminal secretory signal for C4-LCBH.

Nucleic acids encoding both full-length and amino-terminal truncated derivatives of Arabidopsis C4 long chain base hydroxylase were isolated for molecular cloning and gene expression. Total RNA was collected from 14-day-old Arabidopsis thaliana seedlings using published protocol and reagents (Trizol) from Life Technologies, Inc. (Rockville, Md.). 1 μg of total RNA was incubated with 10 pmol of each custom oligos, ATGATGAGTTTCGTGATTTCA (SEQ ID NO:5) and CTCATCTTTGGATACTTTGATTG (SEQ ID NO:6), in a reverse transcription reaction (Thermoscript RT kit, Life Technologies) according to the manufacture's recommendations. Polymerase chain reaction (PCR) was carried out in a total volume of 50 μl with the following reagents: 2 μl of above RT reaction mixture, 20 mM Tris-HCl pH 8.8, 2 mM MgSO₄, 10 mM KCl, 10 mM (NH₄)₂SO₄, 0.1% Triton X-100, 0.1 mg/ml BSA, 10 mM dNTPs, 2.5 units pfu Turbo polymerase (Stratagene, USA), 15 pmol of each of primers SEQ ID NO:7 and SEQ ID NO:8 for full-length LCBH, and 15 pmol of each of primers SEQ ID NO:9 and SEQ ID NO:10 for truncated LCBH.

| | |
|---|---|
| ATATACCTCTATACTTTAACGTCAAGGAGAAAAAAC | SEQ ID NO:7 |
| TATAATGATGAGTTTCGTGATT: | |
| TAACTAATTACATGATATCGACAAAGGAAAAGGGGC | SEQ ID NO:8 |
| CTGTTTAATGATGATGATGATGATGCTCATCTTTGG | |
| ATACTTTG: | |
| ATATACCTCTATACTTTAACGTCAAGGAGAAAAAAC | SEQ ID NO:9 |
| TATAATGTATTTCATTCATCGATATATG: | |
| TAACTAATTACATGATATCGACAAAGGAAAAGGGGC | SEQ ID NO:10 |
| CTGTTTAATGATGATGATGATGATGCTCATCTTTGG | |
| ATACTTTG: | |

PCR cycling was as follows: 94° C. (3 min), 55° C. (1 min), 68° C. (2 min) for 1 cycle, 94° C. (45 sec), 55° C. (30 sec), 68° C. (1 min) for 30 cycles, 68° C. (10 min). The yeast expression vector pYEX19-1 was digested with restriction endonucleases BamHI and SalI as directed by the manufacturer (New England Biolabs, Beverly, Mass.). The resulting PCR products were incorporated into pYEX 19-1 by co-transforming the two linear DNAs into the yeast, Saccharomyces cerevisiae (strain BJ5459, American Type Culture Collection, Manassas, Va.). Colony PCR was performed to identify yeast transformants carrying an intact LCBH gene, indicating recombination between the LCBH PCR product and pYEX 19-1. The Integrity of the above clones was verified by DNA sequence analysis.

### EXAMPLE 7

### Cloning and Expression of Full-Length and Amino-Terminal Truncated Derivatives of Arabidopsis LCBH in a Yeast Sur2 Mutant Strain

The Syringomycin E-resistant sur2 yeast mutant strain (Open Biosystems, cat. no. YSC1021-548967) was aquired to assay for inhibitors of the LCBH protein. SUR2 encodes a hydroxylase involved in sphingolipid metabolism that is highly similar to Arabidopsis LCBH (SEQ ID NO:2) (expect=1E-51, identities=102/252 (40%)) and both proteins hydroxylate sphinganine at the C4 position to yield phytosphingosine. S. cerevisiae sur2 null mutant long chain bases are exclusively dihydrosphingosine, while wild-type cells have phytosphingosine and sphinganine. The sur2 mutant Syringomycin E-resistant phenotype can be suppressed when grown under conditions with exogenous phytosphingosine, thus the formation of phytosphingosine is a prerequisite for Syringomycin E sensitivity.

TABLE 1
Yeast strains used in this study

| Name | Genotype |
|---|---|
| PGY4 | MATa his3Δ200 leu2Δ1 lys2-801 ura3-52 trp1 pep4::HIS3 |
| | prb1Δ1.6R can1 GAL pLCBH-t [pYEX19-1 |
| | (2um_URA3_leu2-d)-GALpro-truncated |
| | LCBH (ppg30610)-CYCter] |
| PGY5 | MATa his3Δ200 leu2Δ1 lys2-801 ura3-52 trp1 pep4::HIS3 |
| | prb1Δ1.6R can1 GAL pLCBH [pYEX19-1 |
| | (2um_URA3_leu2-d)-GALpro-full-length LCBH |
| | (ppg30610)-CYCter] |
| PGY8 | MATa his3Δ1 leu2Δ0 lys2Δ0 ura3Δ0 |
| PGY29 | MATa his3Δ leu2Δ lys2Δ ura3Δ sur2Δ::KanMX |
| PGY30 | MATa his3Δ leu2Δ lys2Δ ura3Δ sur2Δ::KanMX pYEX19-1 |
| PGY31 | MATa his3Δ leu2Δ lys2Δ ura3Δ sur2Δ::KanMX |
| | pLCBH-t (see PGY4) |
| PGY32 | MATa his3Δ leu2Δ lys2Δ ura3Δ sur2Δ::KanMX |
| | pLCBH (see PGY5) |

The yeast sur2 mutant cells were transformed with galactose-inducible yeast expression vectors that express six-histidine tagged derivatives of both full-length and truncated forms of Arabidopsis LCBH (Table 1). Galactose-dependent protein expression was observed in the LCBH transformed strains PGY31, PGY32, PGY4 and PGY5, but not in the vector-only strain PGY30 (data not shown). LCBH degradation products observed in strains PGY31 and PGY32 are likely the result of endogenous yeast proteases that are present in these strains and not due to inherent protein instability (data not shown). When LCBH was expressed in a protease-deficient yeast strain the production of full-length protein improved and the occurrence of degradation products was reduced (data not shown).

Previous studies have shown that Arabidopsis LCBH restores phytosphingosine formation when it is expressed in a yeast sur2 mutant (Sperling P. et al., supra). To confirm production of functional LCBH in the yeast expression strains, long chain base composition was examined by reversed-phase HPLC (data not shown). For the experiments, the yeast extracts were hydrolyzed in 5N NaOH at 110° C. to liberate N-acylated long chain bases. The liberated long chain bases were then converted into dinitrophenyl derivatives to enable elution to be monitored at 350 nm during HPLC. For long chain base extraction yeast strains PGY30, PGY31 and PGY32 were grown for 24 hours in raffinose-containing minimal medium, at which time galactose was added to the cultures to induce expression of the LCBH gene, and the strains were incubated for an additional 24 hours. To enable a semi-quantitative assessment of the long chain base profiles from the three yeast strains, the same wet weight of cells (160 mg) was processed from each strain for HPLC. C₁₈-sphinganine and C₁₈-phytosphingosine (Sigma) were used as derivatization and HPLC standards for this experiment. The sur2 mutant yeast strains expressing either the full-length protein (PGY32) or the truncated form of the protein (PGY31) were able to produce phytosphingosine whereas the non-expressing sur2 mutant (PGY30) was not.

### EXAMPLE 8

### Purification of Syringomycin E

Syringomycin E is not a commercially available antibiotic and was, therefore, purified from the bacterium Pseudomonas syringae pv. syringae according to the method of (Bidwai & Zhang et al., 83 Plant Physiol 39-43 (1987)). Briefly, stationary-phase liquid cultures of P. syringae pv. syringae were extracted with acidified acetone and fractionated over an Amberlite XAD-2 (Sigma) column using a linear gradient of 0.1% trifluoroacetic acid/0.1% trifluoroacetic acid in 2-propanol. Each fraction was assayed for ability to inhibit growth of wild type (PGY8) and sur2 (PGY29) yeast strains. For the assay, 10 ml fractions were collected and 50 μl of each fraction was applied a 96-well plate and dried in a vacuum concentrator. 3001 of rich medium containing mid-logarithmic PGY8 or PGY29 at a cell density of 10⁵ cells/ml were applied to each well. Growth was monitored after 18 hours by determining the absorbance at 600 nm. Syringomycin E-containing fractions were identified as those that inhibited growth of PGY8 but not of PGY29.

### EXAMPLE 9

### Assay for Syringomycin E Sensitivity of Sur2 Mutant Yeast Strains Expressing Full-Length and Amino-Terminal Truncated Derivatives of Arabidopsis LCBH

The ability of the amino-terminal truncated Arabidopsis LCBH to confer Syringomycin E sensitivity on the yeast sur2 mutant was determined as follows. The yeast strain PGY31 (sur2 mutant expressing the truncated form of C4-LCBH) and the control strains PGY8 (wild type) and PGY29 (sur2) were used in the assay. A Syringomycin E-containing fraction from the Amberlite XAD-2 column of Example 8 was evaporated in a vacuum concentrator and resuspended in 50% acetone. The resulting Syringomycin E-containing solution was approximately 14-fold concentrated compared with the fraction recovered from the XAD-2 column. 5 μl of the solution was added to the wells of a 384-well plate and dried. 100 μl of yeast strains PGY8 and PGY29 in rich medium, or PGY31 in rich medium supplemented with galactose were added to each well and growth was monitored at 600 nm. Growth of the PGY8 and PGY31 yeast strains was completely inhibited in the presence of Syringomycin E. Only the Sur2 mutant strain, PGY29, was able to grow in the presence of Syringomycin E, demonstrating that the amino-terminal truncated Arabidopsis LCBH was able to restore Syringomycin E sensitivity to the Sur2 mutant stain.

### EXAMPLE 10

### Assay for the Identification of Inhibitors of LCBH Activity

LCBH inhibitors are identified by measuring a restoration of growth to a heterologous LCBH protein-expressing Sur2 mutant yeast strain in the presence of sufficient Syringomycin E to inhibit growth of a Sur2 mutant yeast strain not expressing the heterologous LCBH protein. Measurement of growth is determined as described in Example 9.

While the foregoing describes certain embodiments of the invention, it will be understood by those skilled in the art that variations and modifications may still fall within the scope of the invention.

What is claimed is:

1. A method for identifying a compound as a candidate for a herbicide, comprising:
a) measuring cell growth of a sur2 strain of mutant yeast cells expressing a heterologous LCBH polypeptide in a media containing syringomycin E in the presence and absence of a compound, wherein the heterologous LCBH polypeptide confers on the mutant yeast cells a sensitivity to syringomycin E and the syringomycin E is present at an amount sufficient to suppress growth of the mutant yeast cells; and
b) comparing the cell growth measurement in the presence and absence of the compound, wherein an increase in growth in the presence, relative to the absence, of the compound indicates the compound as a candidate for a herbicide.

2. The method of claim 1, wherein the heterologous LCBH polypeptide is an Arabidopsis polypeptide.

3. The method of claim 1, wherein the heterologous LCBH polypeptide is the LCBH polypeptide set forth in SEQ ID NO:2 or SEQ ID NO:4.

4. The method of claim 1, wherein the heterologous LCBH polypeptide is a LCBH polypeptide consisting essentially of SEQ ID NO:2.

5. The method of claim 1, wherein the heterologous LCBH polypeptide is selected from the group consisting of:
a) a LCBH polypeptide that is a plant polypeptide;
b) a LCBH polypeptide that is a dicot plant polypeptide;
c) a LCBH polypeptide that is a monocot plant polypeptide;
d) a LCBH polypeptide that is other than a C3 plant polypeptide; and
e) a LCBH polypeptide that is other than a C4 plant polypeptide.

6. The method of claim 1, wherein the heterologous LCBH polypeptide is selected from the group consisting of:
a) a LCBH polypeptide having at least 50% sequence conservation with SEQ ID NO:4 and at least 10% of the activity of SEQ ID NO:2;
b) a LCBH polypeptide comprising at least 50 consecutive amino acids of SEQ ID NO:2 and having at least 10% of the activity of SEQ ID NO:2; and
c) a LCBH polypeptide comprising at least 50 amino acids having at least 50% sequence conservation with SEQ ID NO:2 and having at least 10% of the activity of SEQ ID NO:2.

7. A method for identifying a compound as a candidate for a herbicide, comprising: measuring the activity of a LCBH polypeptide in the presence and absence of a compound, wherein an alteration of the LCBH polypeptide activity in the presence of the compound indicates the compound as a candidate for a herbicide.

8. The method of claim 7, wherein the LCBH polypeptide is an Arabidopsis polypeptide.

9. The method of claim 7, wherein the LCBH polypeptide is the LCBH polypeptide set forth in SEQ ID NO:2 or SEQ ID NO:4.

10. The method of claim 7, wherein the LCBH polypeptide is a LCBH polypeptide consisting essentially of SEQ ID NO:2.

11. The method of claim 7, wherein the LCBH polypeptide is selected from the group consisting of:
a) a LCBH polypeptide that is a plant polypeptide;
b) a LCBH polypeptide that is a dicot plant polypeptide;
c) a LCBH polypeptide that is a monocot plant polypeptide;
d) a LCBH polypeptide that is other than a C3 plant polypeptide; and
e) a LCBH polypeptide that is other than a C4 plant polypeptide.

12. The method of claim 7, wherein the LCBH polypeptide is selected from the group consisting of:
a) a LCBH polypeptide having at least 50% sequence conservation with SEQ ID NO:2 and at least 10% of the activity of SEQ ID NO:2;
b) a LCBH polypeptide comprising at least 50 consecutive amino acids of SEQ ID NO:2 and having at least 10% of the activity of SEQ ID NO:2; and
c) a LCBH polypeptide comprising at least 50 amino acids having at least 50% sequence conservation with SEQ ID NO:2 and having at least 10% of the activity of SEQ ID NO:2.

13. A method for identifying a compound as a candidate for a herbicide, comprising:
a) contacting a LCBH polypeptide with a compound; and
b) detecting the presence or absence of binding between the compound and the LCBH polypeptide, wherein binding indicates that the compound is a candidate for a herbicide.

14. The method of claim 13, wherein the LCBH polypeptide is an Arabidopsis polypeptide.

15. The method of claim 13, wherein the LCBH polypeptide is the LCBH polypeptide set forth in SEQ ID NO:2 or SEQ ID NO:4.

16. The method of claim 13, wherein the LCBH polypeptide is a LCBH polypeptide consisting essentially of SEQ ID NO:2.

17. The method of claim 13, wherein the LCBH polypeptide is selected from the group consisting of:
a) a LCBH polypeptide that is a plant polypeptide;
b) a LCBH polypeptide that is a dicot plant polypeptide;
c) a LCBH polypeptide that is a monocot plant polypeptide;
d) a LCBH polypeptide that is other than a C3 plant polypeptide; and
e) a LCBH polypeptide that is other than a C4 plant polypeptide.

18. The method of claim 13, wherein the LCBH polypeptide is selected from the group consisting of:
a) a LCBH polypeptide having at least 50% sequence conservation with SEQ ID NO:2 and at least 10% of the activity of SEQ ID NO:2;
b) a LCBH polypeptide comprising at least 50 consecutive amino acids of SEQ ID NO:2 and having at least 10% of the activity of SEQ ID NO:2; and
c) a LCBH polypeptide comprising at least 50 amino acids having at least 50% sequence conservation with SEQ ID NO:2 and having at least 10% of the activity of SEQ ID NO:2.

19. A method for identifying a compound as a candidate for a herbicide, comprising:
a) measuring the expression of a LCBH in a plant, or tissue thereof, in the presence and absence of a compound; and
b) comparing the expression of the LCBH in the presence and absence of the compound, wherein an altered expression in the presence of the compound indicates that the compound is a candidate for a herbicide.

20. The method of claim 19, wherein the plant is Arabidopsis.

21. The method of claim 19, wherein the expression of the LCBH is measured by detecting the LCBH mRNA.

22. The method of claim 19, wherein the expression of the LCBH is measured by detecting the LCBH polypeptide.

23. The method of claim 19, wherein the expression of the LCBH is measured by detecting the LCBH polypeptide enzyme activity.

24. An isolated nucleic acid comprising a nucleotide sequence that encodes the polypeptide of SEQ ID NO:4.

25. An isolated nucleic acid comprising a nucleotide sequence that encodes a polypeptide consisting essentially of SEQ ID NO:4.

26. A recombinant polypeptide consisting essentially of the amino acid sequence of SEQ ID NO:4.

27. A recombinant polypeptide comprising the amino acid sequence of SEQ ID NO:4.


SEQUENCE LISTING (10 sequences)

SEQ ID NO: 1 - DNA, 777 bp, Arabidopsis thaliana
atgagtttcgtgatttcagatgaatttctggggacatttgtgccgattctggtgtattgggtgtattcagggatgtacatttgcttaggatctttggataaatatagattgcattcgaagatagatgaggatgagaagaatctcgtttctaaatctgccgtcgttaagggcgttcttcttcaacagactcttcaggctattatctccgttattctcttcaagataacaggaagtgatgcagatgctgctacgacacagcaattttccattctacttctggctaggcagtttatcatcgctatgcttgtaattgacacttggcaatatttcattcatcgatatatgcacctcaacaagttcttatacaagcacatccactctcagcatcatcgccttattgtgccttattcgtatggagctctatacaaccatccactagaaggtcttctcttggacactattggtggtgctttgtctttcctcttctctggtatgtccccaagaacagccatctttttcttctccttcgctaccatcaaaacagtagatgatcattgtggactatggcttccgggaaatccatttcacatcttcttcagtaataactctgcttaccatgatgttcaccaccaactctatggaaccaaatacaacttctcgcagccattctttgttatgtgggataggattctcggcacttacttgccttattcattggagaaaagagccaatggaggatttgaaacacggccaatcaaagtatccaaagatgagtaa

SEQ ID NO: 2 - protein, 258 aa, Arabidopsis thaliana
MSFVISDEFLGTFVPILVYWVYSGMYICLGSLDKYRLHSKIDEDEKNLVSKSAVVKGVLLQQTLQAIISVILFKITGSDADAATTQQFSILLLARQFIIAMLVIDTWQYFIHRYMHLNKFLYKHIHSQHHRLIVPYSYGALYNHPLEGLLLDTIGGALSFLFSGMSPRTAIFFFSFATIKTVDDHCGLWLPGNPFHIFFSNNSAYHDVHHQLYGTKYNFSQPFFVMWDRILGTYLPYSLEKRANGGFETRPIKVSKDE

SEQ ID NO: 3 - DNA, 453 bp, Arabidosis thaliana
tatttcattcatcgatatatgcacctcaacaagttcttatacaagcacatccactctcagcatcatcgccttattgtgccttattcgtatggagctctatacaaccatccactagaaggtcttctcttggacactattggtggtgctttgtctttcctcttctctggtatgtccccaagaacagccatctttttcttctccttcgctaccatcaaaacagtagatgatcattgtggactatggcttccgggaaatccatttcacatcttcttcagtaataactctgcttaccatgatgttcaccaccaactctatggaaccaaatacaacttctcgcagccattctttgttatgtgggataggattctcggcacttacttgccttattcattggagaaaagagccaatggaggatttgaaacacggccaatcaaagtatccaaagatgagtaa

SEQ ID NO: 4 - protein, 150 aa, Arabidopsis thaliana
YFIHRYMHLNKFLYKHIHSQHHRLIVPYSYGALYNHPLEGLLLDTIGGALSFLFSGMSPRTAIFFFSFATIKTVDDHCGLWLPGNPFHIFFSNNSAYHDVHHQLYGTKYNFSQPFFVMWDRILGTYLPYSLEKRANGGFETRPIKVSKDE

SEQ ID NO: 5 - DNA, 21 bp, artificial
  primer
atgatgagtttcgtgatttca

SEQ ID NO: 6 - DNA, 23 bp, artificial
  primer
ctcatctttggatactttgattg

SEQ ID NO: 7 - DNA, 58 bp, artificial
  primer
atatacctctatactttaacgtcaaggagaaaaaactataatgatgagtttcgtgatt

SEQ ID NO: 8 - DNA, 80 bp, artificial
  primer
taactaattacatgatatcgacaaaggaaaaggggcctgtttaatgatgatgatgatgatgctcatctttggatactttg

SEQ ID NO: 9 - DNA, 64 bp, artificial
  primer
atatacctctatactttaacgtcaaggagaaaaaactataatgtatttcattcatcgatatatg

SEQ ID NO: 10 - DNA, 80 bp, artificial; identical to SEQ ID NO: 8
  primer